(19) United States
(12) Patent Application Publication
(10) Pub. No.: US 2015/0335614 A1
(43) Pub. Date: Nov. 26, 2015

(54) COMPOSITIONS FOR REDUCING BETA-AMYLOID-INDUCED NEUROTOXICITY COMPRISING BETA-SECRETASE INHIBITOR

(71) Applicant: Pholoronol, Inc., San Francisco, CA (US)

(72) Inventors: Bong Ho Lee, Daejon (KR); Seongho Kim, Daejon (KR); Hyeon-Cheol Shin, Bonney Lake, WA (US); Haengwo Lee, Bellevue, WA (US)

(21) Appl. No.: 14/817,215

(22) Filed: Aug. 4, 2015

Related U.S. Application Data
(62) Division of application No. 12/948,054, filed on Nov. 17, 2010, now abandoned.
Application No. 12/948,054 is a continuation of application No. 13/796,025, filed on Mar. 12, 2013, now abandoned.

(51) Int. Cl. A61K 31/357; A61K 31/635; A61K 31/05; A61K 31/5513; A61K 31/192; A61K 31/353; C07D 493/04; A61K 31/55

(52) U.S. Cl. CPC A61K 31/357; C07D 493/04; A61K 31/635; A61K 31/55; A61K 31/5513; A61K 31/192; A61K 31/353; A61K 31/05

(57) ABSTRACT

Disclosed are a composition for reducing beta amyloid-induced neurotoxicity by inhibiting β-secretase activity, comprising a dibenzofuran derivative, and a method for preparing the same. Further disclosed is that the combination of the dibenzofuran derivative with a γ-secretase inhibitor or an anti-inflammatory agent shows higher activity with respect to reducing beta amyloid-induced neurotoxicity.

### BACKGROUND OF THE INVENTION

1. Field of the Invention

The present invention relates to a composition for inhibiting β-secretase activity, comprising a dibenzofuran derivative as an active ingredient. Also, the present invention is concerned with a composition for reducing beta amyloid-induced neurotoxicity by inhibiting the production of β-secretase.

2. Description of the Related Art

Beta amyloid (Aβ), which is a peptide consisting of 40-42 amino acids, is formed after the sequential cleavage of the amyloid precursor protein (APP) by β-secretase (BACE, or Beta-site APP-cleaving enzyme) and γ-secretase.

When secreted outside brain cells, the beta amyloid protein (Aβ) progressively forms highly neurotoxic plaques that injure brain cells, thus causing degenerative cognitive impairments such as Alzheimer's disease.

In detail, β-secretase cleaves the amyloid precursor protein, a transmembrane protein of neurons, at the β-cleavage site to form the N-terminus of beta-amyloid, followed by cleavage of γ-secretase at the γ-cleavage site within the membrane region of APP to produce the C-terminal end of the beta amyloid protein. The beta amyloid proteins are thus separated from the membrane aggregate into oligomers which undergo fibrilization to form highly neurotoxic plaque deposits.

Over recent decades, a variety of methods for effectively reducing the secretion of beta amyloid have arisen as core targets for developing therapeutic drugs for diseases associated therewith.

Accordingly, antibodies against beta amyloid for neutralizing the toxicity of beta amyloid, and materials for inhibiting the production of amyloid precursor proteins have been researched and developed, but still not yet put into practice owing to insufficient clinical efficacy and major side effects.

Extensive research and clinical studies have been done to target γ-secretase, which is involved in the final step of the beta amyloid production pathway, so as to develop an agent for reducing the secretion of the neurotoxic β-amyloid protein. As a result, γ-secretase inhibitors were developed and have been under a lot of clinical investigation, but most of them are known to exhibit insufficient medicinal efficacy with major side effects. Particularly, γ-secretase inhibitors also inhibit the Notch signaling pathway, causing the side effect of interrupting cell-cell communication.

Meanwhile, mice which lack β-secretase, responsible for the first step in the production of beta amyloid from the amyloid precursor protein, that is, β-secretase-knockout mice are known to be healthy without significant side effects. Accordingly, β-secretase inhibitors, which were proven to show less side effects, have been studied as targets for the development of medicaments for reducing beta amyloid secretion.

Among the BACE inhibitors known thus far, there are peptide-based inhibitors similar to physiological substrates and synthetic compounds based on structures suitable for binding to the active site of BACE. However, peptide-based inhibitors lack practicality because their uptake into brain cells when administered orally is difficult. Also, due to concerns about toxicity and side effects, synthetic compounds are anticipated to have little clinical effect in practice when administered over a long period of time.

In addition, commonly used anti-inflammatory agents (e.g., NSAIDs, COX inhibitors, etc.) have been reported to alleviate neurotoxicity. However, since they exhibit significant side effects such as gastrointestinal hemorrhage, thrombosis, upon long-term administration or high dose administration, the use of them alone has not been enough to guarantee successful clinical achievements.

To effectively treat neurodegenerative diseases, there is a need for natural materials or quasi-natural materials that show a minimum of side effects or toxicity in spite of being administered over a long period of time. Almost all existing drugs that have been approved for use in the treatment of neurodegenerative diseases are highly toxic and at most temporally alleviate the symptoms. None of them are known to exert substantial therapeutic effects on neurodegenerative diseases.

### SUMMARY OF THE INVENTION

It is therefore an object of the present invention to provide a composition which is of low cytotoxicity and is capable of inhibiting the production of β-secretase, thus effectively reducing beta-amyloid-induced neurotoxicity.

It is another object of the present invention to provide a composition for reducing beta-amyloid-induced neurotoxicity, which can be used in combination with pre-existing γ-secretase inhibitors so that the dosage and side effects of the pre-existing γ-secretase inhibitors can be minimized, with a concomitant maximal reduction in the beta amyloid level.

It is a further object of the present invention to provide a composition for reducing beta-amyloid-induced neurotoxicity, which can be used in combination with anti-inflammatory agents having a neurotoxicity reducing function so that the dosage and side effects can be minimized, with a concomitant maximal reduction in the beta amyloid levels.

The objects of the present invention could be accomplished by a provision of a composition for inhibiting β-secretase activity, comprising as an active ingredient a dibenzofuran derivative represented by the following Chemical Formula 3 or 4 or a mixture thereof.

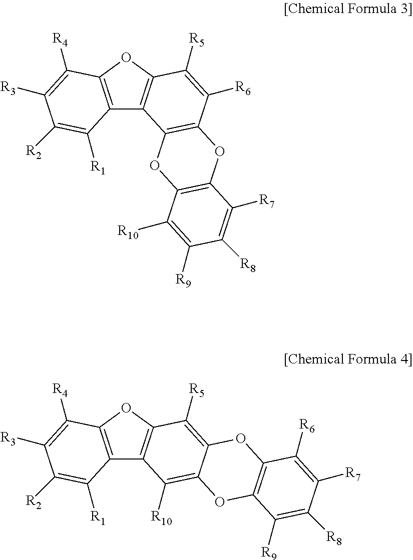

wherein,

R₁ to R₁₀ are each independently selected from among H, OH, OMe and 3,5-dihydroxyphenoxyl of the following Chemical Formula 2.

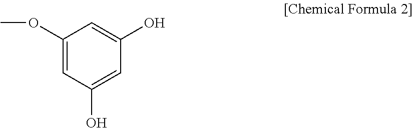

Therefore, in accordance with an aspect thereof, the present invention provides a composition for reducing beta amyloid-induced neurotoxicity, comprising the dibenzofuran derivative of Chemical Formula 3 or 4 or a mixture thereof as an active ingredient responsible for the inhibition of β-secretase activity.

In accordance with another aspect thereof, the present invention provides a composition for reducing beta amyloid-induced neurotoxicity, comprising a mixture of a dibenzofuran derivative selected from among the compound of Chemical Formula 3, the compound of Chemical Formula 4, and a mixture thereof, and a γ-secretase inhibitor or an anti-inflammatory compound at a weight ratio of from 0.1:99.9 to 99.9:0.1.

In accordance with a further aspect thereof, the present invention provides a method for preventing and treating mild-cognitive impairment and Alzheimer's disease using a composition for reducing beta amyloid-induced neurotoxicity comprising the dibenzofuran derivative of Chemical Formula 3 or 4 or a mixture thereof as an active ingredient responsible for the inhibition of β-secretase activity.

Having effective inhibitory activity against β-secretase, the composition comprising the dibenzofuran derivative in accordance with the present invention can reduce the neurotoxicity induced by beta amyloid, one of the principal causes of neurodegenerative diseases, effectively and safely.

In addition, the composition for reducing neurotoxicity according to the present invention can be used in combination with γ-secretase inhibitor or an anti-inflammatory agent, which is difficult to apply to clinical practice due to its high toxicity and side effects as well as low clinical effects, so as to induce a synergistic effect far superior to the neurotoxicity reducing effects obtained by using them individually.

### DESCRIPTION OF THE PREFERRED EMBODIMENTS OF THE INVENTION

In accordance with an aspect thereof, the present invention pertains to a composition for inhibiting β-secretase activity, comprising as an active ingredient the dibenzofuran derivative of the following Chemical Formula 3 or 4 or a mixture thereof.

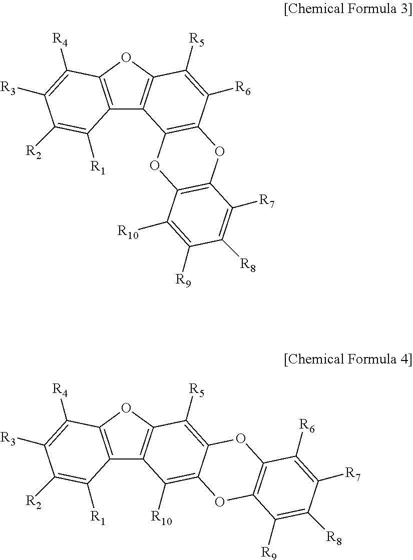

wherein,

R₁ to R₁₀ are each independently selected from among H, OH, OMe and 3,5-dihydroxyphenoxyl of the following Chemical Formula 2.

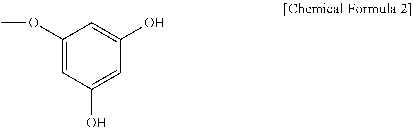

In Chemical Formula 3 or 4, at least one of the substituents R₄, R₇ and R₉ is preferably the 3,5-dihydroxyphenoxyl of Chemical Formula 2.

In accordance with another aspect thereof, the present invention pertains to a composition for reducing beta amyloid-induced neurotoxicity, comprising the dibenzofuran derivative of Chemical Formula 3 or 4 or a mixture thereof as an active ingredient responsible for the inhibition of β-secretase activity.

The dibenzofuran derivatives represented by Chemical Formula 3 or 4 may be synthesized using typical organic chemistry or may be obtained by extraction from brown algae, for example, Eisenia arborea, Ecklonia radiata, Eisenia bicyclis, Ecklonia kurome, Ecklonia cava, Ecklonia stolonifera, or Ecklonia maxima.

In the composition for inhibiting β-secretase activity or for reducing beta amyloid-induced neurotoxicity, the active ingredient including the dibenzofuran derivative may be used in an amount of from 0.01 to 50 wt % based on the total weight of the composition. A nanomolar concentration of the dibenzofuran derivative of the present invention is sufficient to show inhibitory activity against beta secretase.

In accordance with another aspect thereof, the present invention pertains to a composition for reducing beta amyloid-induced neurotoxicity, comprising a combination of a dibenzofuran derivative selected from among compounds of Chemical Formulas 3, the compound of Chemical Formula 4, and a mixture thereof, and a γ-secretase inhibitor or an anti-inflammatory compound as an active ingredient inhibitory of β-secretase activity.

In the composition for reducing beta amyloid-induced neurotoxicity, the active ingredient including a combination of the dibenzofuran derivative and the γ-secretase inhibitor or anti-inflammatory compound may be contained in an amount of from 0.01 to 50 wt % based on the total weight of the composition.

In the composition for reducing beta amyloid-induced neurotoxicity, the dibenzofuran derivative of Chemical Formula 3 or 4 or a mixture thereof may be in mixture with a γ-secretase inhibitor or an anti-inflammatory compound at a weight ratio of from 0.1:99.9 to 99.9:0.1, and preferably at a weight ratio of from 9:1 to 99.9:0.1.

Examples of the γ-secretase inhibitor useful in the art include, but are not limited to, (S)-2-[2-(3,5-Difluoro-phenyl)-acetylamino]-N—((S)-5-methyl-6-oxo-6,7-dihydro-5H-dibenzo[b,d]azepin-7-yl)-propionamide of the following Chemical Formula 5:

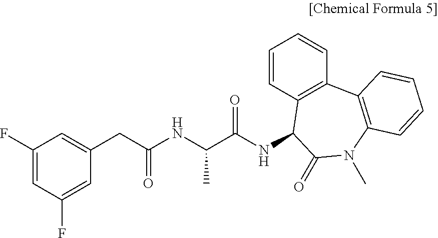 [SMILES: C[C@H](NC(=O)Cc1cc(F)cc(F)c1)C(=O)N[C@@H]1C(=O)N(C)c2ccccc2-c2ccccc21]

### (S)-2-[2-(3,5-Difluoro-phenyl)-acetylamino]-N—((S)-1-methyl-2-oxo-5-phenyl-2,3-dihydro-1H-benzo[e][1,4]diazepin-3-yl)-propionamide of the following Chemical Formula 6

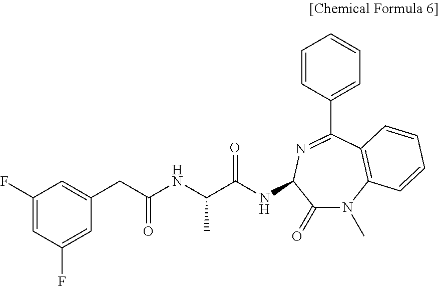 [SMILES: C[C@H](NC(=O)Cc1cc(F)cc(F)c1)C(=O)N[C@H]1N=C(c2ccccc2)c2ccccc2N(C)C1=O]

### N-[(3,5-Difluorophenyl)acetyl]-L-alanyl-2-phenyl]glycine-1,1-dimethylethyl ester of the following Chemical Formula 7

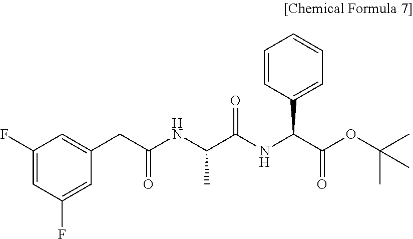 [SMILES: C[C@H](NC(=O)Cc1cc(F)cc(F)c1)C(=O)N[C@H](C(=O)OC(C)(C)C)c1ccccc1]

Among the anti-inflammatory compounds useful in the present invention are NSADIS (Non-Steroidal Anti-inflammatory Drugs) including non-selective COX (cyclooxygenase) inhibitors and selective COX-2 (cyclooxygenase-2) inhibitors, and natural polyphenols such as quercetin, curcumin, catechin and resveratrol, but the present invention is not limited thereto. Examples of the non-selective COX inhibitors include ibuprofen, naproxen, diclofenac, and aspirin while celicoxib, rofecoxib and valsecoxib fall within the range of the selective COX-2 inhibitors.

In accordance with a further aspect thereof, the present invention pertains to a method for the prevention and treatment of mild-cognitive impairment and Alzheimer's disease using a composition for reducing beta amyloid-induced neurotoxicity comprising a dibenzofuran derivative of Chemical Formula 3 or 4 or a mixture thereof as an active ingredient responsible for inhibitory activity against β-secretase.

In order to prevent or treat neurodegenerative diseases such as mild-cognitive impairment and Alzheimer's disease, production of beta amyloids (1-42) should be suppressed in the cerebral nerve cells which are subjected to the condition of amyloid precursor protein overexpression. When applied to nerve cells overexpressing APP, the composition comprising the dibenzofuran derivative of Chemical Formula 3 or 4 or a mixture thereof was found to inhibit beta amyloid activity far more efficiently than did the preexisting representative compounds known for inhibitory activity against beta amyloid.

In addition, experiments for evaluating inhibitory activity against beta amyloid (1-42) production and effects on neurotoxicity reduction showed that far greater effects were obtained when a composition comprising the dibenzofuran derivative of Chemical Formula 3 or 4 or a mixture and a preexisting γ-secretase inhibitor or anti-inflammatory agent at various mixture ratios was used than when the composition comprising the dibenzofuran derivative of Chemical Formula 3 or 4 or a mixture thereof or the preexisting compound was used separately.

A better understanding of the present invention may be obtained through the following examples which are set forth to illustrate, but are not to be construed as limiting the present invention.

### EXAMPLES

### Preparation Example 1

### Preparation of Primary 1,3,5-Trihydroxybenzene Polymer Product

300 g of 1,3,5-trihydroxybenzene was subject to polymerization at 230° C. for 1 hr under the condition of a water content of 5, 10, 20 or 50 wt % and a pressure of 0.1, 1, 3, 10 or 100 mmHg Each product was extracted with 1 L of 80% ethanol to remove insoluble matter therefrom, followed by drying in a vacuum to give a black solid. The solid was washed with distilled water and recovered in a n-butanol layer to yield primary 1,3,5-trihydroxybenzene polymer products.

### Experimental Example 1

### Assay of Primary 1,3,5-Trihydroxybenzene Polymer Product for Inhibitory Activity Against B-Secretase

The primary 1,3,5-trihydroxybenzene polymer products obtained under the various conditions were assayed for inhibitory activity against β-secretase. For this purpose, a human recombinant BACE1 assay kit (PanVera, WI, USA) was used according to the manufacturer's instructions.

10 μL of a substrate (75 μM Rh-EVNLDAEFK-Quencher in 50 mM ammonium bicarbonate) was mixed with 10 μL of the enzyme (BACE1, 1 U/mL), 10 μL of an assay buffer, 10 μL of a sample solution (a solution of the primary 1,3,5-trihydroxybenzene polymer product in an assay buffer) to give a reaction mixture in which the sample was contained in a concentration of 1 mg/mL. For a negative control, an assay buffer containing none of the samples was used instead of the sample solution. In the absence of light, each reaction mixture was allowed to react at 25° C. for 60 min. Thereafter, while a light beam at 528 nm was applied to the reaction mixture, fluorescent light at 620 nm was detected with Bio-Tek Microplate fluorescence reader FLx 800 (VT, USA) Inhibition activity was calculated according to the following formula:

Inhibition (%)=[1−{(S−S₀)/(C−C₀)}]×100

wherein

C is a fluorescent intensity after reaction of the negative control for 60 min,

C₀ is an initial fluorescent intensity of the negative control,

S is a fluorescent intensity after reaction of each sample for 60 min, and

S₀ is an initial fluorescent intensity of each sample.

The primary 1,3,5-trihydroxybenzene polymer product prepared under the condition of a water content of 10 wt % and a pressure of 3 mmHg in Preparation Example 1 was found to exhibit the most potential inhibitory activity against β-secretase as measured by the assay method and was called mix sample #1.

TABLE 1
Inhibitory Activity (%) of the Primary Trihydroxybenzene
Polymer Products Prepared Under the Conditions of
Preparation Example 1 against B-secretase

Water (Wt %)

| mmHg | 5 | 10 | 20 | 50 |
|---|---|---|---|---|
| 0.1 | 25% | 33% | 21% | 23% |
| 1 | 40% | 63% | 55% | 19% |
| 3 | 65% | 92% (Mix sample #1) | 70% | 41% |
| 10 | 38% | 52% | 22% | 8% |
| 100 | 28% | 27% | 16% | 7% |

### Preparation Example 2

### Separation of Dibenzofuran Derivative from Mix Sample #1

50 g of the mix sample #1 was loaded onto a liquid chromatography column Liquid chromatography was performed by eluting with a linear gradient of from 15% to 70% methanol solution over 30 min at a flow rate of 10 mL/min on an HP ODS Hypersil column to obtain 11 main fractions (fraction #1-1 to #1-11). The 11 main fractions, each adjusted to a final concentration of 10 μg/mL, were screened for 50% or higher BACE inhibition. Fraction #1-8 (3.5 g) was measured to show the highest inhibitory activity. In order to identify the components of fraction #1-8, 100 mg of fraction #1-8 was subjected to HPLC [Waters Spherisorb S10 ODS2 column (20×250 mm), eluent: 30% MeOH, flow rate: 3.5 ml/min] to separate four active substances. Their structures were determined by 500 MHz ¹H- and 125 MHz ¹³C-NMR spectrometry (JEOL ECP-500 FT-NMR, JEOL, Japan) and FABMS (VG Autospec Ultima mass spectrometer), with TMS used as the internal standard. The active substances were identified as dibenzofuran derivatives of Chemical Formulas 1-1 to 1-4, respectively. Each compound at a concentration of 10 μg/mL was measured to exhibit 50% or higher β-secretase inhibition (Table 2).

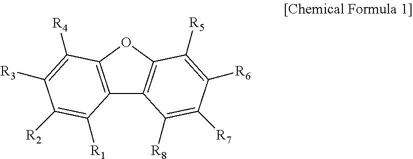

TABLE 2

| | | BACE |
|---|---|---|
| Compound | R group | Inhibition % |
| Chemical | R₁, R₃, R₆, R₈ = OH; R₄, R₅, R₇ = H; | 56% |
| Formula 1-1 | R₂ = 3,5-dihydroxyphenoxyl | |
| Chemical | R₁, R₃, R₆, R₈ = OH; R₂, R₅, R₇ = H; | 66% |
| Formula 1-2 | R₄ = 3,5-dihydroxyphenoxyl | |
| Chemical | R₁, R₃, R₆, R₈ = OH; R₅, R₇ = H | 82% |
| Formula 1-3 | R₂, R₄ = 3,5-dihydroxyphenoxyl | |
| Chemical | R₁, R₃, R₅, R₈ = OH; R₅, R₇ = H; | 87% |
| Formula 1-4 | R₂, R₄ = 3,5-dihydroxyphenoxyl | |

### Preparation Example 3

### Preparation of Secondary 1,3,5-Trihydroxybenzene Polymer Product

To obtain substances which exhibit higher inhibitory activity against β-secretase, the components of fraction #1-8 were further polymerized to produce secondary 1,3,5-trihydroxybenzene polymer products. In this regard, 1000 mL of distilled water, 8.2˜82.0 mM (1.0˜10.0 CMC) of the surfactant sodium dodecyl sulfate (SDS) and 20˜2000 mg of 1,3,5-trihydroxybenzene were added to 200 mg of the fraction #1-8 separated in Preparation Example 2, followed by stirring at 40° C. for 5 hrs under a pressure of 10 mmHg After vacuum evaporation of the solvent, the residue was extracted with 80% methanol to remove insoluble material, and then dried in a vacuum to give a black solid. The solid was washed with distilled water to remove the surfactant and unreacted 1,3,5-trihydroxybenzene, followed by extraction with n-butanol to afford secondary 1,3,5-trihydroxybenzene polymer products.

TABLE 3
Inhibitory Activity (%) of the Secondary 1,3,5-Trihydroxybenzene
Polymer Products against B-secretase According to the Conditions
of Reaction between Fraction #1-8 and 1,3,5-Trihydroxybenzene

| SDS Concentration | Wt. Ratio of 1,3,5-trihydroxybenzene | | | |
|---|---|---|---|---|
| (mM) | 0.1 | 0.5 | 2 | 10 |
| 0 | 0 | 2 | 3 | 5 |
| 1.0 CMC (8.2 mM) | 60 | 72 | 54 | 26 |
| 2.5 CMC (20.5 mM) | 65 | 92 (Mix Sample #2) | 67 | 36 |
| 5.0 CMC (40.1 mM) | 44 | 86 | 55 | 20 |
| 10.0 CMC (82 mM) | 27 | 53 | 35 | 5 |

The secondary 1,3,5-trihydroxybenzene polymer product prepared from fraction #1-8 of Preparation Example 2 reacted with 1,3,5-trihydroxybenzene at the weight ratio of 1:0.5 under the conditions set forth at an SDS concentration of 2.5 CMC (20.5 mM) was found to exhibit the most potential inhibitory activity against β-secretase as measured by the assay method at 1 mg/mL and was called mix sample #2.

### Preparation Example 4

### Separation of Dibenzofuran Derivative from Mix Sample #2

Before identifying compounds which are superior in inhibitory activity, 150 mg of the mix sample #2 was loaded onto a liquid chromatography column Liquid chromatography was performed by eluting with a linear gradient of from 15% to 70% methanol solution over 30 min at a flow rate of 10 mL/min on an HP ODS Hypersil column to obtain 5 main fractions (fraction #2-1 to #2-5). The 5 main fractions, each adjusted to a final concentration of 100 ng/mL, were screened for 50% or higher BACE inhibition. Measurements showed that fractions #2-4 (31 mg) and #2-5 (41 mg) had the highest inhibitory activity.

In order to identify the components of fractions #2-4 and #2-5, 30 mg of fraction #2-4 and 40 mg of fraction #2-5 were subjected to HPLC [Waters Spherisorb S10 ODS2 column (20×250 mm), eluent: 30% MeOH, flow rate: 3.5 ml/min] to elute six and four active substances, respectively. Their structures were determined by 500 MHz ¹H- and 125 MHz ¹³C-NMR spectrometry (JEOL ECP-500 FT-NMR, JEOL, Japan) and FABMS (VG Autospec Ultima mass spectrometer), with TMS used as the internal standard. The active substances were identified as dibenzofuran derivatives having the structures in common with Chemical Formulas 3 and 4, respectively.

### Experimental Example 2

### Assay of Dibenzofuran Derivatives of Chemical Formula 3 and 4 for Inhibitory Activity Against B-Secretase

Each of the dibenzofuran derivatives at a concentration of 10 nM was measured to exhibit 50% or higher β-secretase inhibition (Table 4). The benzofuran derivatives of the present invention were found to exhibit inhibitory activity against beta secretase at lower concentration than the compound of Comparative Example 1 which is known as a potential β-secretase inhibitor (J. Med. Chem. 2004, 47, 6447-6450) as measured by IC50 assay.

TABLE 4

| | | BACE | |
|---|---|---|---|
| | | Inhibition at | IC50 |
| Compound | R group | 10 nM | (nM) |
| Chemical | R₁, R₃, R₆, R₈, R₁₀ = OH; R₂, R₄, R₅, R₇, | 53% | 5.8 |
| Formula 3-1 | R₉ = H | | |
| Chemical | R₁, R₃, R₆, R₈, R₁₀ = OH; R₂, R₅, R₇, R₉ = H; | 87% | 1.7 |
| Formula 3-2 | R₄ = 3,5-dihydroxyphenoxyl | | |
| Chemical | R₁, R₃, R₆, R₈, R₁₀ = OH; R₂, R₅, R₉ = H; R₄, | 85% | 2.2 |
| Formula 3-3 | R₇ = 3,5-dihydroxyphenoxyl | | |
| Chemical | R₁, R₃, R₆, R₈, R₁₀ = OH; R₂, R₄, R₅, R₉ = H; | 90% | 1.5 |
| Formula 3-4 | R₇ = 3,5-dihydroxyphenoxyl | | |
| Chemical | R₁, R₃, R₆, R₈, R₁₀ = OH; R₂, R₅, R₉ = H; R₄, | 96% | 1.1 |
| Formula 3-5 | R₇ = 3,5-dihydroxyphenoxyl | | |
| Chemical | R₁, R₃, R₆, R₈, R₁₀, = OH; R₂, R₅, R₇ = H; R₄, | 94% | 1.3 |
| Formula 3-6 | R₉ = 3,5-dihydroxyphenoxyl | | |
| Chemical | R₁, R₃, R₆, R₈, R₁₀ = OH; R₂, R₄, R₅, R₇, | 65% | 4.7 |
| Formula 4-1 | R₉ = H | | |
| Chemical | R₁, R₃, R₆, R₈, R₁₀ = OH; R₂, R₅, R₇, R₉ = H; | 77% | 3.6 |
| Formula 4-2 | R₄ = 3,5-dihydroxyphenoxyl | | |
| Chemical | R₁, R₃, R₆, R₈, R₁₀ = OH; R₂, R₄, R₅, R₇ = H; | 70% | 4.0 |
| Formula 4-3 | R₉ = 3,5-dihydroxyphenoxyl | | |
| Chemical | R₁, R₃, R₆, R₈, R₁₀ = OH; R₂, R₅, R₇ = H; | 86% | 2.4 |
| Formula 4-4 | R₄, R₉ = 3,5-dihydroxyphenoxyl | | |
| C. Ex. 1 | 1,3-Benzenedicarboxamide, N-[(1S,2R)-3- | 32% | 14.7 |
| | (cyclopropylamino)-2-hydroxy-1- | | |
| | (phenylmethyl)propyl]-5- | | |
| | [methyl(methylsulfonyl)amino]-N′-[(1R)-1- | | |
| | phenylethyl]* | | |

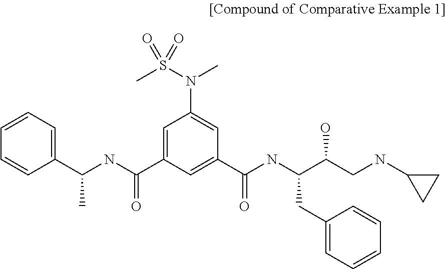 [SMILES: C[C@@H](NC(=O)c1cc(C(=O)N[C@@H](Cc2ccccc2)[C@H](O)CNC2CC2)cc(N(C)S(C)(=O)=O)c1)c1ccccc1]

### Experimental Example 3

### Assay for Inhibitory Effects on the Production of Beta Amyloid in Nerve Cell

N2a/APP cells, used as a study model for neurodegenerative diseases (Wang X C, Zhang Y C, Chatterjie N, Grundke-Iqbal I, Iqbal K, Wang J Z. Neurochem Res (2008) 33:1138-1144), were employed to analyze the inhibition of beta amyloid (Aβ) production in nerve cells. N2a/APP cells were fixed onto 24-well plates (5×10⁴ cells/well) containing a medium devoid of hygromycin B. The ingredients listed in Table 5 were added at a final concentration of 10 μg/mL to each well and incubated for 48 hrs. Secreted Aβ(1-42) was quantitatively analyzed with an Aβ(1-42) immunoassay kit (Biosource, Camarillo, Calif., USA). In this regard, the medium in each well was transferred into 96-well plates coated with an Aβ(1-42) antibody and then treated with a detection antibody. For this, after incubation at room temperature for 3 hrs, the medium was treated with HRP (horseradish peroxidase), an anti-rabbit antibody and stabilized chromogen to allow an antigen-antibody reaction. This reaction was terminated with a stop buffer before absorbance was read at 450 nm. For a negative control, no samples were added to the medium Inhibitory effects on beta amyloid production were calculated according to the following equation and the results are summarized in Table 5, below.

Aβ Inhibition (%)=(C−S)/C×100

where C is the absorbance of a negative control and S is the absorbance of each sample.

### Experimental Example 4

### Reduction of Neurotoxicity

To examine whether the compounds of the present invention are able to reduce the neurotoxicity induced by beta amyloid overexpression, cell viability was assessed. For this, N2a/APP cells were subjected to an MIT assay, which is a measure of mitochondrial activity that converts 3-(4,5-dimethyl-thiazol-2-yl)-2,5-diphenyl-tetrazolium bromide (MTT) to formazan in living cells. N2a/APP cells were seeded at a density of 1×10⁴ cells/well into 96-well plates, incubated for 24 hrs, and left for 12 hrs in serum-free DMEM-Opti-MEM. Thereafter, the samples listed in Table 5 were added at a final concentration of 10 μg/mL to each well (medium free of any of the samples was used for a negative control) and then incubated for 12 hrs. To each well was added approx. 10 μL of MTT (5 mg/ml in phosphate buffered saline (PBS) solution), followed by incubation at 37° C. for 4 hrs. Each well was measured for absorbance at 570 nm Inhibitory activity against neurotoxicity was assessed by an increase rate of cell viability as calculated by the following equation. The results are summarized in Table 5, below.

Increase Rate of Cell Viability (%)=(S−C)/C×100

when C is the absorbance of a negative control and S is the absorbance of each sample.

TABLE 5
Aβ Inhibition Rate and Increased Rate of Cell
Viability in N2a/APP Cells at 10 μg/mL

| | | | Increase |
|---|---|---|---|
| | | Aβ | Rate of |
| | | Inhibition | Cell Vi- |
| Ex. No. | Compound | Rate(%) | ability(%) |
| Ex. 1 | Chemical Formula 3-5, 100 wt % | 32 | 24 |
| Ex. 2 | Chemical Formula 3-6, 100 wt % | 30 | 22 |
| Ex. 3 | Chemical Formula 4-4, 100 wt % | 27 | 21 |
| Ex. 4 | Chemical Formula 3-5, 1 wt %, | 23 | 12 |
| | Ingredient 1, 99 wt % | | |
| Ex. 5 | Chemical Formula 3-5, 1 wt %, | 25 | 3 |
| | Ingredient 2, 99 wt % | | |
| Ex. 6 | Chemical Formula 3-5, 1 wt %, | 22 | 9 |
| | Ingredient 3, 99 wt % | | |
| Ex. 7 | Chemical Formula 3-5, 1 wt %, | 23 | 17 |
| | Ingredient 4, 99 wt % | | |
| Ex. 8 | Chemical Formula 3-5, 1 wt %, | 21 | 14 |
| | Ingredient 5, 99 wt % | | |
| Ex. 9 | Chemical Formula 3-5, 1 wt % | 17 | 15 |
| | Ingredient 6, 99 wt % | | |
| Ex. 10 | Chemical Formula 3-5, 10 wt %, | 45 | 27 |
| | Ingredient 1, 90 wt % | | |
| Ex. 11 | Chemical Formula 3-5, 10 wt %, | 38 | 20 |
| | Ingredient 2, 90 wt % | | |
| Ex. 12 | Chemical Formula 3-5, 10 wt %, | 47 | 28 |
| | Ingredient 3, 90 wt % | | |
| Ex. 13 | Chemical Formula 3-5, 10 wt %, | 42 | 33 |
| | Ingredient 4, 90 wt % | | |
| Ex. 14 | Chemical Formula 3-5, 10 wt %, | 39 | 30 |
| | Ingredient 5, 90 wt % | | |
| Ex. 15 | Chemical Formula 3-5, 10 wt %, | 43 | 38 |
| | Ingredient 6, 90 wt % | | |
| Ex. 16 | Chemical Formula 3-5, 50 wt %, | 65 | 60 |
| | Ingredient 1, 50 wt % | | |
| Ex. 17 | Chemical Formula 3-5, 50 wt %, | 45 | 27 |
| | Ingredient 2, 50 wt % | | |
| Ex. 18 | Chemical Formula 3-5, 50 wt %, | 58 | 33 |
| | Ingredient 3, 50 wt % | | |
| Ex. 19 | Chemical Formula 3-5, 50 wt %, | 62 | 49 |
| | Ingredient 4, 50 wt % | | |
| Ex. 20 | Chemical Formula 3-5, 50 wt %, | 66 | 56 |
| | Ingredient 5, 50 wt % | | |
| Ex. 21 | Chemical Formula 3-5, 50 wt %, | 70 | 65 |
| | Ingredient 6, 50 wt % | | |
| Ex. 22 | Chemical Formula 3-5, 90 wt %, | 55 | 44 |
| | Ingredient 1, 10 wt % | | |
| Ex. 23 | Chemical Formula 3-5, 90 wt %, | 67 | 35 |
| | Ingredient 2, 10 wt % | | |
| Ex. 24 | Chemical Formula 3-5, 90 wt %, | 78 | 44 |
| | Ingredient 3, 10 wt % | | |
| Ex. 25 | Chemical Formula 3-5, 90 wt %, | 54 | 35 |
| | Ingredient 4, 10 wt % | | |
| Ex. 26 | Chemical Formula 3-5, 90 wt %, | 55 | 40 |
| | Ingredient 5, 10 wt % | | |
| Ex. 27 | Chemical Formula 3-5, 90 wt %, | 60 | 47 |
| | Ingredient 6, 10 wt % | | |
| Ex. 28 | Chemical Formula 3-5, 99 wt %, | 40 | 31 |
| | Ingredient 1, 1 wt % | | |
| Ex. 29 | Chemical Formula 3-5, 99 wt %, | 38 | 28 |
| | Ingredient 2, 1 wt % | | |
| Ex. 30 | Chemical Formula 3-5, 99 wt %, | 43 | 30 |
| | Ingredient 3, 1 wt % | | |
| Ex. 31 | Chemical Formula 3-5, 99 wt %, | 37 | 26 |
| | Ingredient 4, 1 wt % | | |
| Ex. 32 | Chemical Formula 3-5, 99 wt %, | 39 | 28 |
| | Ingredient 5, 1 wt % | | |
| Ex. 33 | Chemical Formula 3-5, 99 wt %, | 40 | 30 |
| | Ingredient 6, 1 wt % | | |
| Ex. 34 | Chemical Formula 4-4, 1 wt %, | 25 | 13 |
| | Ingredient 1, 99 wt % | | |
| Ex. 35 | Chemical Formula 4-4, 1 wt %, | 28 | 15 |
| | Ingredient 2, 99 wt % | | |
| Ex. 36 | Chemical Formula 4-4, 1 wt %, | 24 | 10 |
| | Ingredient 3, 99 wt % | | |
| Ex. 37 | Chemical Formula 4-4, 1 wt %, | 25 | 18 |
| | Ingredient 4, 99 wt % | | |
| Ex. 38 | Chemical Formula 4-4, 1 wt %, | 23 | 14 |
| | Ingredient 5, 99 wt % | | |
| Ex. 39 | Chemical Formula 4-4, 1 wt %, | 19 | 16 |
| | Ingredient 6, 99 wt % | | |
| Ex. 40 | Chemical Formula 4-4, 10 wt %, | 50 | 29 |
| | Ingredient 1, 90 wt % | | |
| Ex. 41 | Chemical Formula 4-4, 10 wt %, | 39 | 21 |
| | Ingredient 2, 90 wt % | | |
| Ex. 42 | Chemical Formula 4-4, 10 wt %, | 52 | 30 |
| | Ingredient 3, 90 wt % | | |
| Ex. 43 | Chemical Formula 4-4, 10 wt %, | 46 | 35 |
| | Ingredient 4, 90 wt % | | |
| Ex. 44 | Chemical Formula 4-4, 10 wt %, | 40 | 29 |
| | Ingredient 5, 90 wt % | | |
| Ex. 45 | Chemical Formula 4-4, 10 wt %, | 47 | 41 |
| | Ingredient 6, 90 wt % | | |
| Ex. 46 | Chemical Formula 4-4, 50 wt %, | 72 | 64 |
| | Ingredient 1, 50 wt % | | |
| Ex. 47 | Chemical Formula 4-4, 50 wt %, | 50 | 29 |
| | Ingredient 2, 50 wt % | | |
| Ex. 48 | Chemical Formula 4-4, 50 wt %, | 64 | 35 |
| | Ingredient 3, 50 wt % | | |
| Ex. 49 | Chemical Formula 4-4, 50 wt %, | 68 | 52 |
| | Ingredient 4, 50 wt % | | |
| Ex. 50 | Chemical Formula 4-4, 50 wt %, | 73 | 45 |
| | Ingredient 5, 50 wt % | | |
| Ex. 51 | Chemical Formula 4-4, 50 wt %, | 77 | 70 |
| | Ingredient 6, 50 wt % | | |
| Ex. 52 | Chemical Formula 4-4, 90 wt %, | 61 | 47 |
| | Ingredient 1, 10 wt % | | |
| Ex. 53 | Chemical Formula 4-4, 90 wt %, | 74 | 37 |
| | Ingredient 2, 10 wt % | | |
| Ex. 54 | Chemical Formula 4-4, 90 wt %, | 68 | 35 |
| | Ingredient 3, 10 wt % | | |
| Ex. 55 | Chemical Formula 4-4, 90 wt %, | 59 | 37 |
| | Ingredient 4, 10 wt % | | |
| Ex. 56 | Chemical Formula 4-4, 90 wt %, | 61 | 43 |
| | Ingredient 5, 10 wt % | | |
| Ex. 57 | Chemical Formula 4-4, 90 wt %, | 66 | 43 |
| | Ingredient 6, 10 wt % | | |
| Ex. 58 | Chemical Formula 4-4, 99 wt %, | 44 | 33 |
| | Ingredient 1, 1 wt % | | |
| Ex. 59 | Chemical Formula 4-4, 99 wt %, | 42 | 30 |
| | Ingredient 2, 1 wt % | | |
| Ex. 60 | Chemical Formula 4-4, 99 wt %, | 43 | 33 |
| | Ingredient 3, 1 wt % | | |
| Ex. 61 | Chemical Formula 4-4, 99 wt %, | 41 | 28 |
| | Ingredient 4, 1 wt % | | |
| Ex. 62 | Chemical Formula 4-4, 99 wt %, | 43 | 30 |
| | Ingredient 5, 1 wt % | | |
| Ex. 63 | Chemical Formula 4-4, 99 wt %, | 25 | 13 |
| | Ingredient 6, 1 wt % | | |
| C. Ex. 2 | Ingredient 1 | 21 | 5 |
| C. Ex. 3 | Ingredient 2 | 22 | −5 |
| C. Ex. 4 | Ingredient 3 | 17 | 2 |
| C. Ex. 5 | Ingredient 4 | 19 | 15 |
| C. Ex. 6 | Ingredient 5 | 17 | 12 |
| C. Ex. 7 | Ingredient 6 | 13 | 10 |
| (—) Control | — | 0 | 0 |

* Ingredient 1: (S)-2-[2-(3,5-Difluoro-phenyl)-acetylamino]-N-((S)-5-methyl-6-oxo-6,7-dihydro-5H-dibenzo[b,d]azepin-7-yl)-propionamide(γ-secretase inhibitor, Axon Medchem,
USA)
* Ingredient 2: (S)-2-[2-(3,5-Difluoro-phenyl)-acetylamino]-N-((S)-1-methyl-2-oxo-5-phenyl-2,3-dihydro-1H-benzo[e][1,4]diazepin-3-yl)-propionamide (γ-secretase inhibitor, Axon Medchem, USA)
* Ingredient 3: ibuprofen (anti-inflammatory agent with non-selective COX inhibition function)
* Ingredient 4: celecoxib (anti-inflammatory agent with selective COX-2 inhibition function)
* Ingredient 5: quercetin (natural flavonoid-based anti-inflammatory agent)
* Ingredient 6: resveratrol (natural stilbene-based anti-inflammatory agent)

As described hitherto, the dibenzofuran derivatives of Chemical Formula 3 or 4 in accordance with the present invention are found to exhibit excellent activity with respect to inhibiting the production of beta amyloid and protecting nerve cell, as measured by an immunoassay and an MTT assay. Further, a higher activity of inhibiting the production of beta amyloid and increasing nerve cell viability against beta amyloid-induced neurotoxicity can be attained when the dibenzofuran derivative Chemical Formula 3 or 4, and a γ-secretase inhibitor or an anti-inflammatory agent are used in combination than when used individually.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1-9. (canceled)

10. A composition comprising a mixture of compounds of Chemical Formula 3 and Chemical Formula 4: 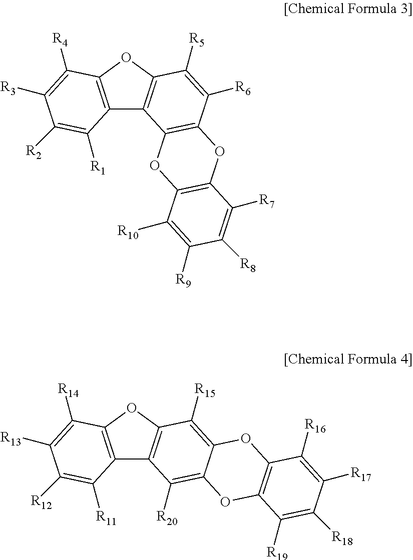
wherein R₁-R₂₀ are independently H, OH, OMe or 3,5-dihydroxyphenoxyl.

11. The composition of claim 10, wherein the composition comprises a mixture of compounds of Chemical Formula 5 and Chemical Formula 6: 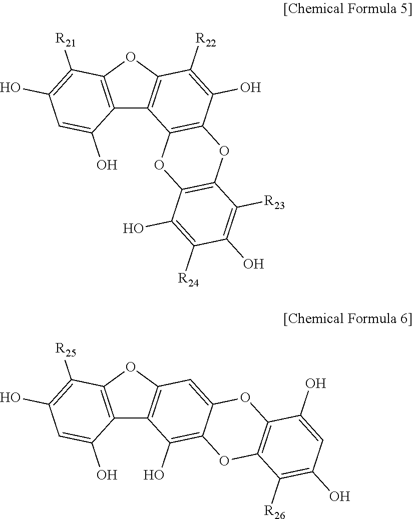
wherein R₂₁-R₂₆ are independently hydrogen or 3,5 dihydroxyphenoxyl.

12. The composition of claim 11, wherein R₂₁ and R₂₃ are 3,5 dihydroxyphenoxyl and R₂₂ and R₂₄ are hydrogen.

13. The composition of claim 11, wherein R₂₅ and R₂₆ are 3,5 dihydroxyphenoxyl.

14. The composition of claim 11, wherein the R₂₁ and R₂₃ are 3,5 dihydroxyphenoxyl, R₂₂ and R₂₄ are hydrogen and R₂₅ and R₂₆ are 3,5 dihydroxyphenoxyl.

15. The composition of claim 11, wherein R₂₁-R₂₄ are hydrogen.

16. The composition of claim 11, wherein R₂₅ and R₂₆ are hydrogen.

17. The composition of claim 11, wherein R₂₁ is 3,5 dihydroxyphenoxyl and R₂₂-R₂₄ are hydrogen.

18. The composition of claim 11, wherein R₂₃ is 3,5 dihydroxyphenoxyl and R₂₁, R₂₂ and R₂₄ are hydrogen.

19. The composition of claim 11, wherein R₂₄ is 3,5 dihydroxyphenoxyl and R₂₁-R₂₃ are hydrogen.

20. The composition of claim 10, wherein the composition further comprises a γ-secretase inhibitor, an anti-inflammatory compound, or a combination thereof.

21. The composition of claim 20, wherein the mixture of compounds of Chemical Formula 3 and Chemical Formula 4 and the γ-secretase inhibitor, the anti-inflammatory compound, or the combination thereof are present in a weight ratio of from 0.1:99.9 to 99.9:0.1 or in a weight ratio of from 9:1 to 99.9:0.1.

22. The composition of claim 20, wherein the γ-secretase inhibitor is selected from the group consisting of (S)-2-[2-(3,5-Difluoro-phenyl)-acetylamino]-N—((S)-5-methyl-6-oxo-6,7-dihydro-5H-dibenzo[b,d]azepin-7-yl)-propionamide, (S)-2-[2-(3,5-Difluoro-phenyl)-acetylamino]-N—((S)-1-methyl-2-oxo-5-phenyl-2,3-dihydro-1H-benzo[e][1,4]diazepin-3-yl)-propionamide and N-[(3,5-Difluorophenyl)acetyl]-L-alanyl-2-phenyl]glycine-1,1-dimethylethyl ester.

23. The composition of claim 20, wherein the anti-inflammatory compound is selected from the group consisting of ibuprofen, naproxen, diclofenac, aspirin, celicoxib, rofecoxib, valsecoxib, quercetin, curcumin, catechin and resveratol.

24. A composition comprising a compound of Chemical Formula 3: 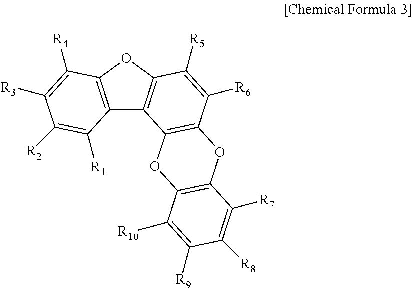
wherein R₁-R₁₀ are independently H, OH, OMe or 3,5-dihydroxyphenoxyl and a γ-secretase inhibitor, an anti-inflammatory compound, or a combination thereof.

25. The composition of claim 24, wherein the compound of Chemical Formula 3 and the γ-secretase inhibitor, the anti-inflammatory compound, or the combination thereof are present in a weight ratio of from 0.1:99.9 to 99.9:0.1 or in a weight ratio of from 9:1 to 99.9:0.1.

26. A composition comprising a compound of Chemical Formula 4: 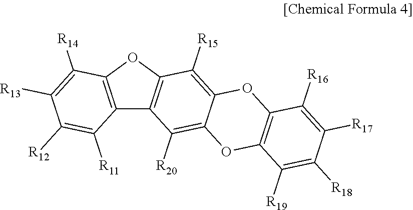
wherein R₁₁-R₂₀ are independently H, OH, OMe or 3,5-dihydroxyphenoxyl.

27. The composition of claim 26, wherein the composition further comprises a γ-secretase inhibitor, an anti-inflammatory compound, or a combination thereof.

28. The composition of claim 27, wherein the compound of Chemical Formula 4 and the γ-secretase inhibitor, the anti-inflammatory compound, or the combination thereof are present in a weight ratio of from 0.1:99.9 to 99.9:0.1 or in a weight ratio of from 9:1 to 99.9:0.1.
